(19) United States
(12) Patent Application Publication
(10) Pub. No.: US 2022/0210628 A1
(43) Pub. Date: Jun. 30, 2022

(54) WEARABLE NETWORKING AND ACTIVITY MONITORING DEVICE

(71) Applicant: Lindelani Mfundo Xulu KA DLAMINI, Pretoria (ZA)

(72) Inventor: Lindelani Mfundo Xulu KA DLAMINI, Pretoria (ZA)

(21) Appl. No.: 17/608,272

(22) PCT Filed: Mar. 4, 2020
(86) PCT No.: PCT/IB2020/051828

§ 371 Date: Nov. 2, 2021

(30) Foreign Application Priority Data
May 2, 2019 (ZA) 2019/02770

(51) Int. Cl. H04W 4/80; A61B 5/11; A61B 5/00; A61B 5/0531; A61B 5/0205; A61B 5/021; A61B 5/024; A61B 5/08; A61B 5/145; A61B 5/1455; A61B 5/369; A61B 5/389; H02J 7/00; H02J 7/34; A41D 1/00

(52) U.S. Cl. CPC H04W 4/80; H04W 84/12; A61B 5/6804; A61B 5/0022; A61B 5/0024; A61B 5/742; A61B 5/0531; A61B 5/02055; A61B 5/021; A61B 5/02416; A61B 5/02438; A61B 5/0816; A61B 5/14532; A61B 5/14551; A61B 5/369; A61B 5/389; A61B 5/7475; A61B 5/002; A61B 5/1112; H02J 7/0048; H02J 7/342; H02J 7/0042; A41D 1/002; A61B 5/1118

(57) ABSTRACT

This invention relates to a wearable networking and activity monitoring device. According to the invention there is provided a wearable networking and activity monitoring device comprising a wearable garment, electronic circuitry comprising at least one conductive member integrally formed with the wearable garment and a network router attached to the garment, the network router configured to operatively broadcast a wireless local area network around the garment enabling a user to connect multiple mobile devices to the Internet via the wireless local area network. The wearable networking and activity monitoring device further comprising a power supply means, which is electrically coupled to the mobile network router through the electronic circuitry and allows the user of the garment to charge the mobile device, and an activity monitoring device embedded in the garment and operable to monitor activity and physiological information of the user.

### FIELD OF THE INVENTION

This invention relates to a wearable networking and activity monitoring device. More specifically but not exclusively, the invention relates to a wearable garment which in use is able to broadcast a personal hotspot to allow a user to connect multiple mobile devices to the Internet, secure personal data, as well as monitoring the activity of the wearer.

### BACKGROUND OF THE INVENTION

In today's age of connectivity, smartphones and tablets are becoming more of a necessity rather than a luxury. Whether it is not to miss important emails, being updated with the news or finding directions when lost—connecting to the Internet anytime and anywhere is becoming an integral part of our lives.

Users of mobile electronic devices are increasingly relying on continuous access to Wide Area Networks, (WAN), such as the world wide web, (www), Internet and Intranets via wireless local area networks. Wireless communication systems utilizing designated radio frequency bands have allowed mobile devices to interconnect with the Internet whenever the mobile device is within the usable range of a compatible wireless network.

Conventional wireless local area networks, (WLAN) are often deployed inside structures such as homes, offices and public and commercial buildings for networking with client mobile computers and other clients' mobile electronic devices. In addition, larger wireless network deployment over a campus or city is also known to network with a large number of client mobile computers and other clients' mobile electronic devices.

Recently mobile network routers that provide a personal wireless local area network (hotspot) which enables a user to connect multiple electronic devices to the internet are becoming increasingly popular. These devices can, however, become an inconvenience as they need to be carried by the user. Other disadvantages include inadequate network coverage and limited battery life.

At the same time, corporate and personal data such as documents, presentations, music, gaming, videos, etc. are stored and carried in portable USB flash drives, memory cards, hard drives which have a Universal Serial Bus (USB) port to connect to other devises such as computers and laptops. These unfortunately tend to be easily misplaced, leading to loss of important data. It has been recognised that a need exists to have a manner of portable data storage that will not easily get misplaced whilst on the move.

It has long been recognised that a need exists for a user to have a constant personal wireless local area network while on the move with adequate coverage without the inconvenience which is caused by current mobile network routers.

### OBJECT OF THE INVENTION

It is accordingly an object of the present invention to provide a device that provides a user with a continuous personal wireless local area network with which the applicant believes the aforementioned limitations and disadvantages may at least be alleviated or which would provide useful alternatives for known devices.

### SUMMARY OF THE INVENTION

According to a first aspect of the invention, there is provided a wearable networking and activity monitoring device comprising;
a wearable garment;
electronic circuitry comprising at least one conductive member integrally formed with the wearable garment;
a network router attached to the garment, the network router configured to operatively broadcast a wireless local area network around the garment enabling a user to connect multiple mobile devices to the Internet via the wireless local area network;
power supply means electrically coupled to the mobile network router through the electronic circuitry; the power supply means configured to operatively allow the user of the garment to charge the mobile device; and
an activity monitoring device embedded in the garment comprising;
   a plurality of sensors, disposed in the garment, to generate sensor data which is representative of activity and/or physiological information of the user of the garment;
   a processor in communication with the plurality of sensors, to calculate the activity and/or physiological information from the generated sensor data; and
   a visual display device, to display the activity and/or physiological information of the user.

The wearable networking and activity monitoring device may include an embedded data storage device. The data storage device may be electrically coupled with the power supply means and have a connectivity port on the outer layer of the jacket. The connectivity port may be in the form of anyone USB, Firewire, eSata, or the like allowing a user to operatively transfer data from an external source to the data storage device. In another embodiment of the invention the data storage device may have a wireless communication module in the form of a Bluetooth module allowing a user to transfer data wirelessly to the data storage device. The data storage device may be in the form of a hard drive which is embedded in the garment.

The wearable garment may be in the form of any article of clothing which is intended to be worn on the body of the user. In a preferred embodiment, the wearable garment is in the form of a jacket.

The jacket may comprise an internal layer defining an inner surface of the jacket and an external layer defining an outer surface of the jacket. The external layer may be made from any suitable material with the internal layer in the form of a lining stitched to the external layer.

In another embodiment, the internal layer may be in the form of a secondary jacket which is removably secured to the external layer by means of snap fasteners, Velcro, a clasp locker or the like.

The electronic circuitry may comprise conductive members in the form of conductive cables which are disposed between the inner surface and the outer surface of the jacket.

In yet another embodiment the jacket may be in the form of a singular layer having an inner surface and an outer surface. In such an embodiment the conductive members may be in the form of conductive yarns which are woven into the jacket.

The power supply means may be in the form of a rechargeable battery. The power supply means may include a battery charger having at least one input that is operatively connectable to an external power source. In a preferred embodiment, the power supply means may be in the form of a power bank comprising a rechargeable battery and a battery charger. The power bank may be in the form of any suitable power bank known in the art.

The power bank may include at least one output through which the user can connect and charge the mobile device. The output may be electrically coupled with an outlet port on the outer surface of the jacket. The outlet port may be in the form of a USB port which in use can receive a mobile device charger. In another embodiment, the power bank may include inductive charging means. The inductive charging means may allow a user to charge the mobile device by placing the device near the power bank. In such an embodiment the jacket may include a mobile device holder in which the mobile phone can be placed while charging.

The battery charger of the power bank includes an input to operatively receive a charging cable from an external power source to recharge the power bank. In a preferred embodiment, the input of the power bank may be electrically coupled to a charging port located on the outer surface of the jacket. In use, a user may electrically couple an external power source to the charging port to recharge the power bank without removing it from the jacket.

The rechargeable power bank may be detachably attached to the outer surface of the jacket allowing a wearer to remove it when needed. In a preferred embodiment, the power bank may be detachably secured between the inner layer and outer layer of the jacket.

The wearable networking and activity monitoring device may include flexible photovoltaic panels located on the external layer of the jacket. The flexible photovoltaic panels may be electrically coupled to the power bank and operatively recharge the power bank.

The plurality of sensors of the activity monitoring device may include an altimeter with the generated sensor data in the form of a change in an altitude of the user. The processor is operable to calculate the number of stairs or flights of stairs that the user has climbed from the change in the altitude.

The plurality of sensors may further include a motion sensor in the form of an accelerometer and a pedometer with the generated sensor data in the form of the movement of the wearer. The processor is operable to calculate the number of steps and/or distance that the user has walked/ran.

The plurality of sensors may also include a physiological sensor with the generated sensor data representative of the physiological information of the user. The physiological sensor may be in the form of a bioimpedance sensor, optical sensor or the like. The processor is operable to calculate the blood pressure, heart rate, respiration rate, skin conduction, blood glucose, blood oxygenation, skin temperature, body temperature, electromyography and/or electroencephalography of the user using the generated physiological information.

The activity monitoring device may include data storage means electrically coupled with the processor. The data storage means operatively stores the data relating to the activity and/or physiological information of the user. The stored data may subsequently be retrieved and presented to the user via the visual display device.

The activity monitoring device may include communication circuitry to communicate with an external user interface, the Internet, a social media site, computing system or the like. The communication circuitry may be in the form of a wireless, optical or wired module. In a preferred embodiment, the communication circuitry may be linked with the network router allowing the activity monitoring device to connect to the mobile device connected to the wireless local area network. In another embodiment, the communication circuitry is in the form of a Bluetooth module. The communication circuitry may be configured to share the physiological information of the user with a pre-selected third party when the physiological information of the user is outside a pre-set safety range.

The activity tracker may include a rechargeable battery, in a preferred embodiment, the activity tracker may be electrically coupled with the power supply means.

The wearable networking and activity monitoring device may further include permanently fixed or detachable antennas that are electrically connected to the network router. The permanently fixed or detachable antennas may be in the form of a transmit and a receive antenna which in use increase the signal strength and area of the wireless local area network. The permanently fixed or detachable antennas may be embedded between the inner surface and the outer surface of the jacket. In another embodiment, the permanently fixed or detachable antennas may extend outward from the outer surface of the jacket.

The wearable networking and activity monitoring device may include a sim card port, in connection with the network router, which is embedded in the outer surface of the jacket.

The network router may further include an Ethernet port which is embedded in the outer surface of the jacket.

The wearable networking and activity monitoring device may be equipped with one or more buzzers and/or speakers with which to alert the user. The device may buzz or emit a sound in order to notify the user that the battery level of the power bank is low.

The wearable networking and activity monitoring device may also be equipped with one or more visual indicators with which to indicate information to the user. The one or more visual indicators may be located on the outer surface of the jacket and indicate the battery level of the power bank, the wireless local area network strength and the like.

The wearable networking and activity monitoring device may also include a global positioning system tracker with which the location of the wearable networking and activity monitoring device can be monitored. The global positioning system tracker may be configured to send the location of the user to a selected third party. The wearable networking and activity monitoring device may include an emergency button enabling the location of the user to be send to a third party.

The wearable networking and activity monitoring device may further include a panic button which in use, and upon activation by the user is operable to broadcast a emergency signal to alert people near the user.

### BRIEF DESCRIPTION OF THE ACCOMPANYING DRAWING

The invention will now further be described, by way of an example only, with reference to the accompanying FIGURE wherein:

FIG. 1 is a three-dimensional view of a wearable networking and activity monitoring device according to one aspect of the invention.

### DESCRIPTION OF A PREFERRED EMBODIMENT OF THE INVENTION

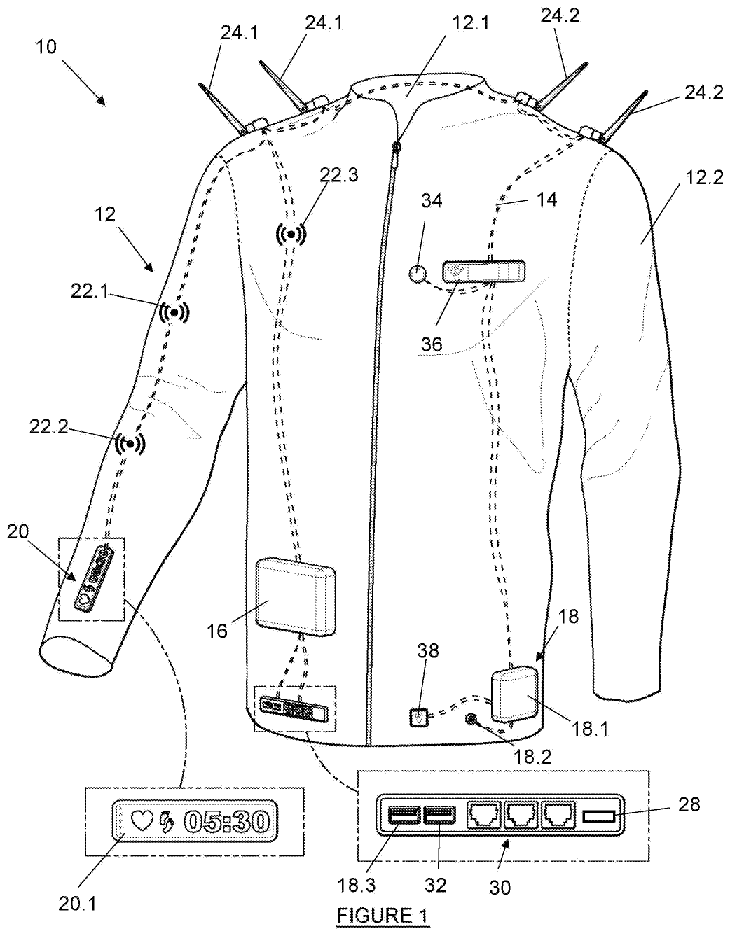

A wearable networking and activity monitoring device is generally indicated by reference numeral 10 in FIG. 1. The wearable networking and activity monitoring device 10 comprises a garment 12 in the form of a jacket which is intended to be worn on the body of the user (not shown). The wearable networking and activity monitoring device 10 includes electronic circuitry 14 comprising a plurality of conductive members 14 integrally formed with the jacket 12.

The jacket is in the form of a single layer jacket with an inner surface and an outer surface. The conductive members 14 of the electronic circuitry are in the form of conductive yarns which are woven into the jacket 12.

In another embodiment that is not shown the jacket comprises multiple layers with an internal layer defining the inner surface of the jacket and an external layer defining an outer surface of the jacket. In such an embodiment the external layer of the jacket in the form of leather, cotton, polyester or any other suitable material with the internal layer in the form of a lining stitched to the external layer. Alternatively, the internal layer is in the form of a secondary jacket which is removably secured to the external layer by means of snap fasteners, Velcro, clasp locker or the like. In such an embodiment the conductive members of the electronic circuitry are in the form of conductive cables that are disposed between the internal layer and the external layer of the jacket.

The wearable networking and activity monitoring device 10 includes a network router 16 embedded in the jacket 12. The network router 16 is removably secured to the outer surface 12.2 of the jacket. The network router 16 is configured to operatively broadcast a wireless local area network. In use, a user can connect multiple mobile devices (not shown) to the Internet through the broadcasted wireless local area network.

The wearable networking and activity monitoring device 10 further includes power supply means 18. The power supply means 18 is electrically coupled to the network router 16 via the conductive members 14 and operatively capable to provide the network router 16 with power through the electronic circuit 14.

The power supply means 18 is in the form of a power bank comprising a rechargeable battery (not shown) and a battery charger (not shown) disposed in a housing of the power bank 18.1. The power bank 18 includes an input which is electrically coupled with a charging port 18.2 located on the outer surface 12.2 of the jacket. In use, the user is able to connect an external power source (not shown) to the power bank 14 via the charging port 18.2 to recharge the power bank 18 without removing it from the jacket 12.

The power bank 18 is detachably secured to the outer surface of the jacket 12.2 which allows the user to remove the power bank 18 when needed. Preferably the housing 18.1 of the power bank is in the form of a waterproof housing.

The power bank 18 also includes an output (not shown) which allows the user to charge their mobile devices via the power bank 18. The output (not shown) is electrically coupled with an outlet port 18.3 located on the outer surface 12.2 of the jacket 12. The outlet port 18.3 is in the form of a USB ports 18.3 which in use can receive a mobile device charger (not shown). Alternatively, the power bank 18 includes inductive charging means (not shown) which allows a user to charge the mobile device (not shown) by placing the device near the power bank 18.

The power bank 18 is further coupled to flexible photovoltaic panels (not shown) which are secured to the outer surface 12.2 of the jacket 12. In use, the flexible photovoltaic panels (not shown) can recharge the power bank 18 while the user wears the jacket 12.

The wearable networking and activity monitoring device 10 further comprises an activity monitoring device 20. The activity monitoring device 20 comprises a plurality of sensors 22, attached to the jacket 12, to generate sensor data which is representative of activity information and/or physiological information of the user wearing the jacket 12.

The activity monitoring device 20 further comprises a processor (not shown), electrically connected to the plurality of sensors 22. The processor (not shown) is configured to record and calculate activity information and/or physiological information from the generated sensor data.

The plurality of sensors 22 includes an altimeter 22.1 which is configured to generate sensor data representative of the change in the altitude of the user. The processor (not shown) is operable to calculate the number of stairs or flight of stairs the user has climbed from the change in altitude sensed by the altimeter 22.1.

The plurality of sensors 22 also includes a motion sensor 22.2 in the form of an accelerometer and/or pedometer which is configured to generate sensor data representative of the movement of the user. The processor (not shown) is operable to calculate the number of steps and/or the total distance walked/ran by the user from the generated sensor data.

The plurality of sensors 22 further includes a physiological sensor 22.3, attached to the internal layer 12.1 of the jacket 12, which is configured to generate sensor data representative of the physiological condition of the user. The physiological sensor 22.3 is in the form of either a bioimpedance sensor and/or an optical sensor. The processor (not shown) is operable to calculate the blood pressure, heart rate, respiration rate, skin conduction, blood glucose, blood oxygenation, skin temperature, body temperature, electromyography and/or electroencephalography of the user from the generated physiological sensor data.

The activity monitoring device 20 further comprises a visual display device 20.1 in the form of a screen connected to the processor (not shown) The screen 20.1 is located on the outer surface 12.2 of the jacket, to display the calculated physiological information of the user, the calculated number of stairs or flight of stairs the user has climbed, and the calculated number of steps and/or the total distance walked/ran by the user.

The activity monitoring device 20 includes a data storage device (not shown), in communication with the processor (not shown), to store the calculated physiological information of the user, the calculated number of stairs or flight of stairs the user has climbed, and the calculated number of steps and/or the total distance walked/ran by the user. In use, the user may request to view historic data which has been stored on the data storage device.

The activity monitoring device 20 further includes communication circuitry (not shown) allowing the processor to communicate with an external user interface, the Internet, a social media site, computing system or the like. The communication circuitry (not shown) is linked with the network router 16 allowing the activity monitoring device 20 to connect and share stored physiological and/or activity information with the mobile device (not shown) connected to the wireless local area network. The communication circuitry is configured to share the physiological information of the user with a pre-selected third party when the physiological information of the user is outside a pre-set safety range.

The activity monitoring device 20 includes an internal battery (not shown). The activity monitoring device 20 is also connected to the power bank 18 allowing the device 20 to function when the internal battery is drained.

The wearable networking and activity monitoring device includes antennas 24 in electrical communication with the network router 16 and which are attached to the jacket 12. The antennas 24 are in the form of transmit antennas 24.1 and receive antennas 24.2 which in use increase the signal strength and area of the wireless local area network. The antennas 24 are removably attached to the jacket 12 via attachment formations 26 located on the outer surface 12.2 of the jacket. The attachment formations 24.3 are in the form of screw fittings allowing the antennas 24 to be attached and removed as needed.

The wearable networking and activity monitoring device 10 includes a sim card port 28, located on the outer surface 12.2 of the jacket and in communication the network router 16. In use, the user can insert a sim card into the sim card port 28 allowing the network router 16 to transform the 3G/4G or 5G connection or subsequent connection technology of the sim card and broadcast the wireless local area network.

The wearable networking and activity monitoring device 10 also includes a plurality of Ethernet ports 30 which are embedded in the outer surface 12.2 of the jacket. The Ethernet ports 30 in use, allow the user to connect a mobile device to the network router via a wired connection. The Ethernet ports 30 allow the user to connect the network router 16 to a local area network allowing the network router 16 to transmit the local area network over the wireless local area network allowing the mobile devices to wirelessly connect to the local area network.

The wearable networking and activity monitoring device 10 further includes an embedded data storage device (not shown), electrically coupled with the power bank 18. The embedded data storage device (not shown) includes a connectivity port 32 on the outer surface 12.2 of the jacket, in the form of a USB port allowing a user to operatively transfer data from an external source to the data storage device (not shown). The data storage device includes Bluetooth connectivity or other wireless connectivity (not shown) to enable the user to wirelessly transfer of data to and from an external source.

The wearable networking and activity monitoring device is equipped with a power switch 34 with which to turn the network router on and off. The wearable networking and activity monitoring device is further equipped with a buzzer (not shown) with which to alert the user if the battery level of the power bank is low and a visual indicator 36, located on the outer surface 12 of the jacket, with which the battery level of the power bank 18 and the signal strength of the broadcasted wireless local area network are indicated to the user.

The wearable networking and activity monitoring device 10 also includes a global positioning system tracker 38 with which the location of the wearable networking and activity monitoring device 10 can be monitored.

It will be appreciated by those skilled in the art that the invention is not limited to the precise details as described herein and that many variations are possible without departing from the scope and spirit of the invention.

The description above is presented in the cause of providing what is believed to be the most useful and readily understandable description of the principles and conceptual aspects of the invention. In this regard, no attempt is made to show structural details of the invention in more detail than necessary for a fundamental understanding of the invention. The words used should, therefore, be interpreted as words of description rather than words of limitation.

1. A wearable networking and activity monitoring device comprising;
a wearable garment having an inner and an outer surface;
electronic circuitry comprising at least one conductive member integrally formed with the wearable garment;
a network router attached to the wearable garment, and configured to operatively broadcast a wireless local area network around the garment enabling a user to connect multiple mobile devices to the Internet via the wireless local area network;
power supply means electrically coupled to the mobile network router through the electronic circuitry, the power supply means having an output which is electrically coupled with an outlet port, on the outer surface of the garment, configured to operatively receive a charging cable to allow the user of the garment to charge the mobile device; and
an activity monitoring device embedded in the garment comprising;
a plurality of sensors, disposed in the garment, to generate sensor data which is representative of activity and/or physiological information of the user of the garment;
a processor in communication with the plurality of sensors, to calculate the activity and/or physiological information from the generated sensor data; and
a visual display device to display the activity and/or physiological information of the user.

2. (canceled)

3. The wearable networking and activity monitoring device as claimed in claim 1, wherein the power supply means comprises a rechargeable battery and a recharger

4. (canceled)

5. (canceled)

6. The wearable networking and activity monitoring device as claimed in claim 3, wherein the rechargeable battery and recharger defines a power bank which includes an input which is electrically coupled with a charging port, on the outer surface of the garment, to which an external power source is operatively connectable to recharge the battery.

7. (canceled)

8. (canceled)

9. The wearable networking and activity monitoring device as claimed in claim 3, wherein the power bank includes inductive charging means configured to allow a user to charge the mobile device by placing the mobile device near the power bank.

10. (canceled)

11. (canceled)

12. The wearable networking and activity monitoring device as claimed in claim 3, which includes flexible photovoltaic panels, located on the garment's outer surface, and electrically coupled to the recharger of the rechargeable battery.

13. The wearable networking and activity monitoring device as claimed in claim 1, wherein the plurality of sensors include an altimeter with the generated sensor data in the form of a change in an altitude of the user wherein the processor is operable to calculate number of stairs or flight of stairs that the user has climbed from the change in the altitude of the user.

14. (canceled)

15. The wearable networking and activity monitoring device as claimed in claim 1, wherein the plurality of sensors include a motion sensor in the form of an accelerometer and/or a pedometer with the generated sensor data in the form of movement of the user with the processor operable to calculate number of steps and distance that the user has walked/ran.

16. (canceled)

17. (canceled)

18. The wearable networking and activity monitoring device as claimed in claim 1, wherein the plurality of sensors include a physiological sensor selected from any one or more of the group consisting of a bioimpedance sensor and an optical sensor with the generated sensor data representative of physiological information of the user.

19. (canceled)

20. The wearable networking and activity monitoring device as claimed in claim 18, wherein the processor is operable to calculate blood pressure, heart rate, respiration rate, skin conduction, blood glucose, blood oxygenation, skin temperature, body temperature, electromyography and/or electroencephalography of the user using the generated physiological information of the user.

21. (canceled)

22. The wearable networking and activity monitoring device as claimed in claim 1, wherein the activity monitoring device includes communication circuitry to communicate with an external user interface, the Internet, a social media site and a computing system.

23. The wearable networking and activity monitoring device as claimed in claim 10, wherein the communication circuitry is in communication with the network router allowing the activity monitoring device to share information to the mobile device connected to the wireless local area network.

24. The wearable networking and activity monitoring device as claimed in claim 1, which includes antennas electrically connected to the network router.

25. (canceled)

26. (canceled)

27. The wearable networking and activity monitoring device as claimed in claim 1, which includes an Ethernet port and/or a sim card port which is embedded in the outer surface of the garment and in communication with the network router.

28. The wearable networking and activity monitoring device as claimed in claim 1, which includes a data storage device.

29. (canceled)

30. The wearable networking and activity monitoring device as claimed in claim 28, wherein the data storage device includes an input connected to a connectivity port located on the outer layer of the garment allowing the user to transfer data from an external source to the data storage device.

31. The wearable networking and activity monitoring device as claimed in claim 30, wherein the data storage device includes a communication module in the form of a Bluetooth module allowing the user to transfer data from the external source wirelessly to the data storage device.

32. (canceled)

33. The wearable networking and activity monitoring device as claimed in claim 1, which includes a visual indicator with which to indicate a battery level of the power supply means to the user.

34. The wearable networking and activity monitoring device as claimed in claim 1, which includes a global positioning system tracker with which a location of the wearable networking and activity monitoring device can be monitored.

35. The wearable networking and activity monitoring device as claimed in claim 34, which includes an emergency button which upon activation is operable to send the location of the wearable networking and activity monitoring device to a selected third party.

36. The wearable networking and activity monitoring device as claimed in claim 1, wherein the garment is in the form of a jacket with the inner surface defined by the internal layer of the jacket and an outer surface defined by an external layer of the jacket.

37-44. (canceled)
